(19) United States
(12) Patent Application Publication
(10) Pub. No.: US 2017/0312033 A1
(43) Pub. Date: Nov. 2, 2017

(54) SURGERY NAVIGATION SYSTEM

(71) Applicant: METAL INDUSTRIES RESEARCH&DEVELOPMENT CENTRE, Kaohsiung (TW)

(72) Inventors: Bing-Feng HUANG, Kaohsiung City (TW); Wei-Ching WANG, Kaohsiung City (TW); Chih-Lung LIN, Kaohsiung City (TW)

(21) Appl. No.: 15/139,711

(22) Filed: Apr. 27, 2016

(51) Int. Cl. A61B 34/20; A61B 17/70; H04L 27/10; H04B 1/38

(52) U.S. Cl. CPC A61B 34/20; H04L 27/10; H04B 1/38; A61B 17/7077; A61B 2034/2072; A61B 2034/2051

(57) ABSTRACT

A surgery navigation system according to the present invention includes: a positioning module including: a transceiver unit, configured to transmit a frequency modulated signal; multiple positioning marks, which are separately disposed on each vertebra of a spine and configured to transmit the positioning mark frequency signal to the transceiver unit after receiving the frequency modulated signal; and a surgical instrument, which transmits the instrument frequency signal to the transceiver unit after receiving the frequency modulated signal; and a processing unit, which obtains a distance between the positioning marks and the transceiver unit through calculation according to a difference between the positioning mark frequency signal and the frequency modulated signal, and obtains space coordinates of the spine through calculation according to the distance; and obtains a distance between the surgical instrument and the transceiver unit through calculation according to a difference between the instrument frequency signal and the frequency modulated signal, obtains space coordinates of the instrument through calculation according to the distance, and performs a surgery navigation operation according to the space coordinates of the spine and the space coordinates of the instrument.

### BACKGROUND

### Technical Field

The present invention relates to a surgery navigation system, and in particular, to a system in which a difference between a frequency modulated frequency transmitted by a transceiver unit and the frequency modulated frequency reflected by a positioning mark is obtained in a wireless communications manner, a distance between the transceiver unit and the positioning mark is obtained through calculation, and corresponding coordinates are obtained through calculation by using the distance, so as to facilitate a surgery navigation operation.

### Related Art

With the growth of the aging population and influence of life features on modern people, for example, obesity and being sedentary, a disease ratio of a spine increases year by year. If conservative treatment does not work, an implant is generally needed to help a spine to relieve pain and maintain a basic function. The spine has the basic function of protecting the nervous centralis. However, a used implant is rather narrow, for example, a radix arcus vertebrae pedicle nail, and the nervous centralis is damaged even if a slightest mistake occurs.

In the prior art, for example, the U.S. Pat. No. 6,351,659, which is a navigation system patent on a nervous surgery put forward by BrainLab in 1997. The patent basically covers the basis of image guiding surgery technologies, and navigation positioning is based on a combination of infrared and infrared reflection balls. Advantages: Signals are transmitted by using infrared reflection balls; and without influence of a line, there is no obstacle to operations of a surgeon; and without an electronic product, sterilization may be directly performed. Disadvantages: More than three infrared reflection balls are needed to form a coordinate plane; and a distance between balls may not be too small, and otherwise, an excessively large geometry of a DRF affects a surgery.

In addition, for example, the U.S. Pat. No. 8,435,171 discloses an operation interface between a surgeon and an automated assistant, in which positions of a surgical instrument and endoscope support mechanical arms are detected by using a wireless positioning technology, and further a visual field of the endoscope is remotely controlled by tracking the positions of the surgical instrument, thereby implementing a human-machine control function.

In addition, for example, the US patent No. US 20070270660A1 discloses that reflected radio signals are detected by using an antenna array, and a position of a bone of a patient, an implant, or a bone surgical instrument in space is further inferred from sources of the signals.

However, the foregoing technologies do not further disclose how to perform space coordinate transformation and how to apply surgery navigation on a bone of a patient and a medical image.

### SUMMARY

An objective of the present invention is to provide a spine surgery navigation system. A frequency modulated transceiver unit transmits a frequency modulated signal to a positioning mark implanted on each vertebra of a spine, and after the positioning mark reflects the frequency modulated signal back, the distance is obtained through calculation by using a difference between frequencies of the frequency modulated signal when the frequency modulated signal is transmitted and received and coordinate positioning is completed. Therefore, a posture and a position of a spine can be independently tracked and obtained by using a real-time operation, thereby avoiding regarding the spine as a rigid body, improving positioning precision for a vertebra to be operated on, and improving precision and safety for applying a radix arcus vertebrae pedicle nail.

A surgery navigation system for a spine surgery according to the present invention includes a positioning module including a transceiver unit, configured to transmit a frequency modulated signal and receive a positioning mark frequency signal and an instrument frequency signal; multiple positioning marks, which are separately disposed on each vertebra of a spine, where each positioning mark is configured to transmit the positioning mark frequency signal to the transceiver unit after receiving the frequency modulated signal, where the positioning mark frequency signal and the frequency modulated signal are of a same waveform; and a surgical instrument, configured to transmit the instrument frequency signal to the transceiver unit after receiving the frequency modulated signal, where the instrument frequency signal and the frequency modulated signal are of a same waveform; and a processing unit, which obtains a positioning mark distance between the positioning marks and the transceiver unit by using an algorithm through calculation according to a difference between the positioning mark frequency signal and the frequency modulated signal, and obtains space coordinates of the spine through calculation according to the positioning mark distance; and obtains an instrument distance between the surgical instrument and the transceiver unit by using the algorithm through calculation according to a difference between the instrument frequency signal and the frequency modulated signal, obtains space coordinates of the instrument through calculation according to the instrument distance, and performs a surgery navigation operation according to the space coordinates of the spine and the space coordinates of the instrument.

### BRIEF DESCRIPTION OF THE DRAWINGS

The disclosure will become more fully understood from the detailed description given herein below for illustration only, and thus are not limitative of the disclosure, and wherein:

FIG. 1 is a schematic diagram of a surgery navigation system according to the present invention;

FIG. 2 is a block diagram of a surgery navigation system according to the present invention;

FIG. 3A and FIG. 3B is a schematic diagram of a frequency modulated signal of a surgery navigation operation according to the present invention;

FIG. 4 is a schematic diagram of an image of a surgery navigation operation according to the present invention;

FIG. 5 is a schematic diagram of antennas of a positioning mark according to the present invention; and

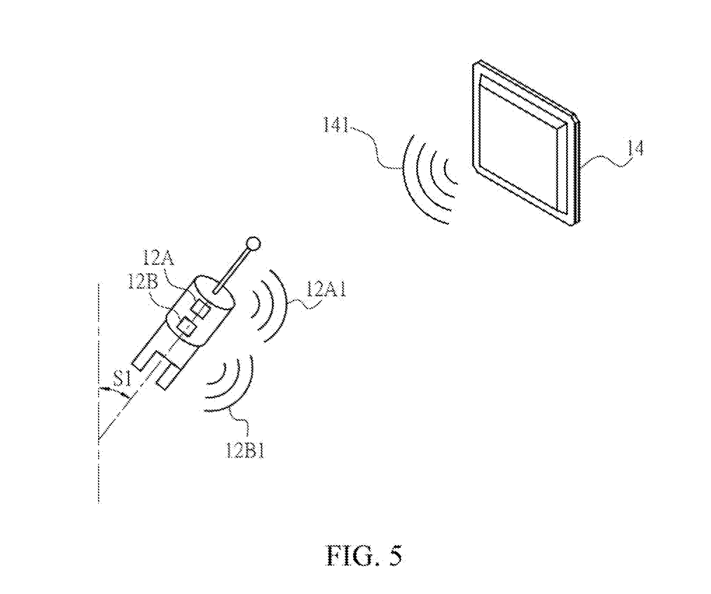

FIG. 6 is a schematic diagram of antennas of a surgical instrument according to the present invention.

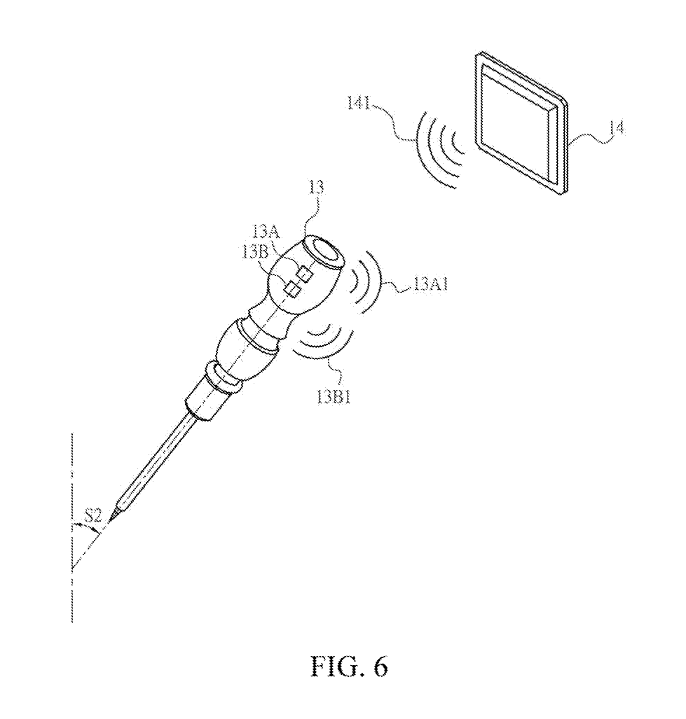

### DETAILED DESCRIPTION

To make the above descriptions of this application and other objectives, features, and advantages clearer, the following makes a detailed description with reference to the accompanying drawings.

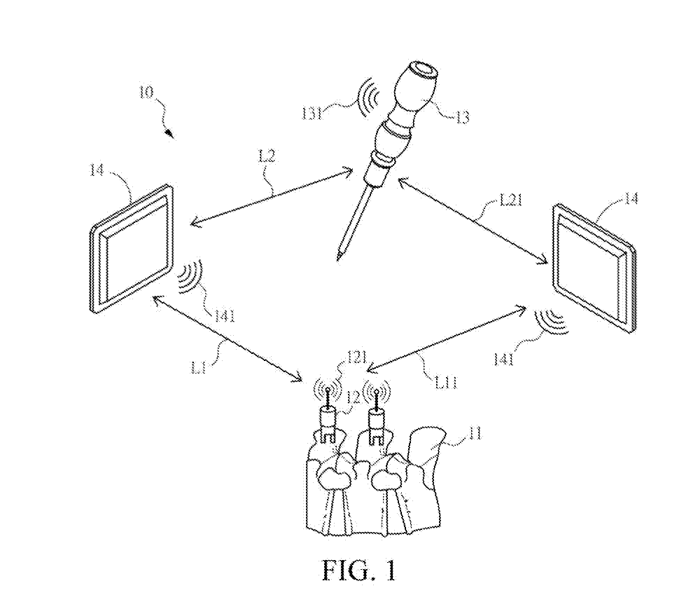

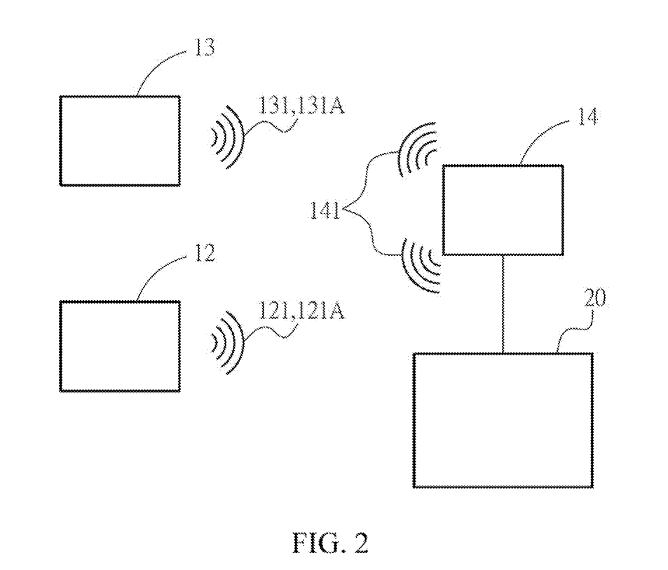

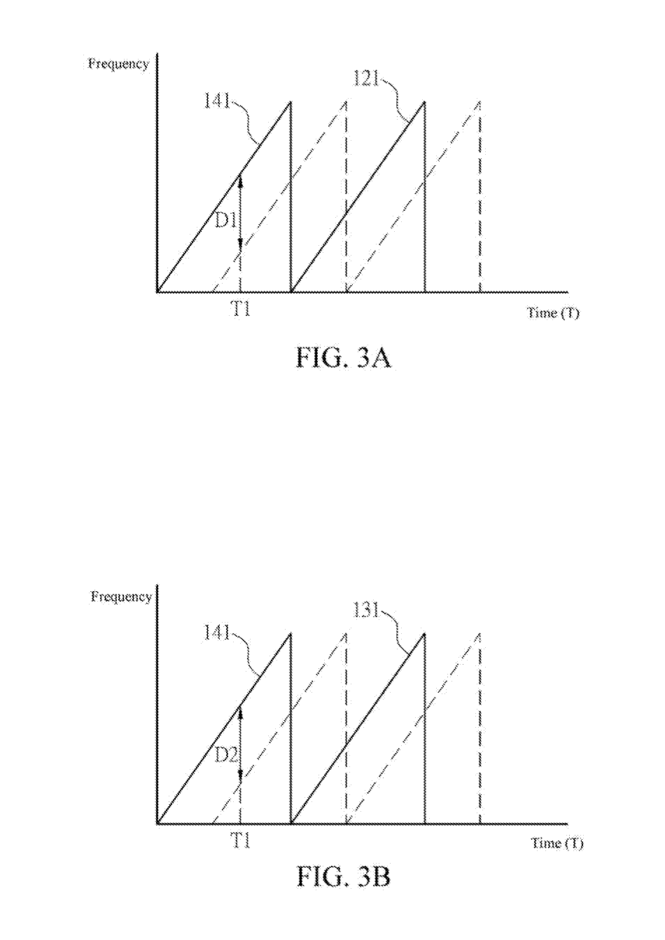

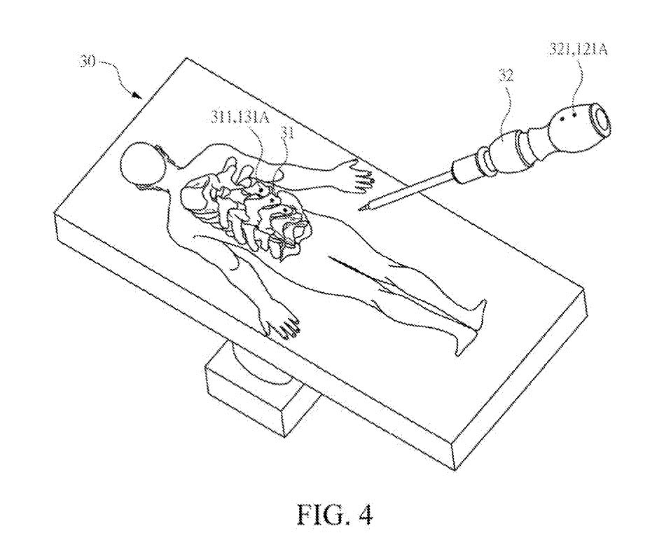

Refer to FIG. 1 to FIG. 4, FIG. 1 is a schematic diagram of a surgery navigation system according to the present invention, FIG. 2 is a block diagram of a surgery navigation system according to the present invention, FIG. 3A and FIG. 3B is a schematic diagram of a frequency modulated signal of a surgery navigation operation according to the present invention, and FIG. 4 is a schematic diagram of an image of a surgery navigation operation according to the present invention. A spine CT image is first shot before a spine surgery, then a C-arm image of the positioning marks 12 that are according to the present invention and that have been implanted inside a spine is taken, and then a surgery image 30 is obtained by overlapping the two images. Therefore, the surgery image 30 may include a spine image 31 and a surgical instrument image 32. Based on this, the surgery navigation system according to the present invention is used to perform a surgery navigation operation. The surgery navigation system according to the present invention includes: a positioning module 10. The positioning module 10 includes a transceiver unit 14, configured to transmit a frequency modulated signal 141 to the multiple positioning marks 12 and a surgical instrument 13.

The multiple positioning marks are separately disposed on each vertebra of a spine 11, where each positioning mark 12 is configured to transmit a positioning mark frequency signal 121 to the transceiver unit 14 after receiving the frequency modulated signal 141. The transceiver unit receives the positioning mark frequency signal 121. The positioning mark frequency signal 121 and the frequency modulated signal 141 are of a same waveform. Specifically, the positioning marks 12 have antennas inside, and after receiving the frequency modulated signal 141, the antenna transmits the signal back to the transceiver unit 14. Therefore, the positioning mark frequency signal 121 and the frequency modulated signal 141 are of a same waveform.

In addition, the surgical instrument 13 is configured to transmit an instrument frequency signal 131 to the transceiver unit 14 after receiving the frequency modulated signal 141. The transceiver unit 14 receives the instrument frequency signal 131. The instrument frequency signal 131 and the frequency modulated signal 141 are of a same waveform. Specifically, the surgical instrument 13 has antennas inside, and after receiving the frequency modulated signal 141, the antenna transmits the signal back to the transceiver unit 14. Therefore, the instrument frequency signal 131 and the frequency modulated signal 141 are of a same waveform.

Electrically connected to the transceiver unit 14, a processing unit 20 obtains a positioning mark distance L1 between the positioning marks 12 and the transceiver unit 14 by using an algorithm through calculation according to a difference D1 between the positioning mark frequency signal 121 and the frequency modulated signal 141, where the algorithm is a frequency modulated continuous wave (FMCW) positioning algorithm, and the processing unit 20 obtains the positioning mark distance L1 through calculation according to the difference D1 between the positioning mark frequency signal 121 and the frequency modulated signal 141 that are received at a same time T1, and obtains space coordinates 311 of the spine according to the positioning mark distance L1.

Specifically, in the above descriptions, because of a fast transmission speed and a small time difference, a same time T1 is used as a sample in the present invention.

Described as above, there are at least two transceiver units 14, which are separately disposed at a periphery of the spine 11. The processing unit obtains two corresponding positioning mark distances L1 and L11 through calculation according to two positioning mark frequency signals 121 received by the two transceiver units 14 and then obtains the space coordinates 311 of the spine through calculation by using the triangulation method.

In addition, the processing unit 20 obtains an instrument distance L2 between the surgical instrument 13 and the transceiver unit 14 by using the FMCW positioning algorithm through calculation according to a difference D2 between the instrument frequency signal 131 and the frequency modulated signal 141, and obtains space coordinates 321 of the instrument through calculation according to the instrument distance L2.

As described above, precisely, there are at least two transceiver units 14, which are separately disposed at a periphery of the surgical instrument 13. The processing unit 20 obtains two corresponding instrument distances L2 and L21 through calculation according to two instrument frequency signals 131 received by the two transceiver units 14 and then obtains the space coordinates 321 of the instrument through calculation by using the triangulation method.

Further, the positioning marks 12 further include an identifier 121A, and the surgical instrument 13 further includes an instrument identifier 131A. The transceiver unit 14 is configured to receive the identifier 121A and the instrument identifier 131A, the processing unit 20 establishes a correspondence between the identifier 121A and the space coordinates 311 of the spine by means of defining, and the processing unit 20 establishes a correspondence between the instrument identifier 131A and the space coordinates 321 of the instrument by means of defining, so that whether a corresponding positioning mark and surgical instrument is correct may be determined by means of the identifier 121A and the instrument identifier 131A.

Further, each positioning mark further includes at least two antennas 12A and 12B. After receiving the frequency modulated signal 141, the at least two antennas transmit two positioning mark frequency signals 12A1 and 12B1 to the transceiver unit 14. The processing unit obtains distances between the two antennas 12A and 12B and the transceiver unit 14 through calculation by using the two positioning mark frequency signals 12A1 and 12B1, so as to obtain an angle S1 of the positioning mark through calculation.

In addition, the surgical instrument 13 further includes at least two instrument antennas 13A and 13B. After receiving the frequency modulated signal 141, the at least two instrument antennas 13A and 13B transmit two instrument frequency signals 13A1 and 13B1 to the transceiver unit 14, and the processing unit obtains distances between the two instrument antennas and the transceiver unit through calculation by using the two instrument frequency signals 13A1 and 13B1, so as to obtain an angle S2 of the surgical instrument through calculation.

As described above, according to the present invention, surgery plan information may be first input before a spine surgery, and then a surgery navigation operation is performed according to the space coordinates of the spine, the space coordinates of the instrument, and the angles of the positioning mark and the surgical instrument.

According to the present invention, a multi-vertebra positioning and tracking navigation surgery technology is implemented by using a wireless positioning technology. With a frequency modulated radio frequency positioning technology and an identifier identification function, a positioning antenna mark is applied to each vertebra to be operated on. Instead of regarding an entire spine as a rigid body in a numeral inferring manner, each vertebra that is operated on is independently tracked, thereby improving the registration precision and speed (which accelerates the calculation convergence) of a medical image and improving application safety and precision of a surgery implant. In addition, the navigation system is applicable to a long-spine surgery (correction of spine scoliosis and fracture of multiple vertebrae), so that an operation is not limited by a large infrared reflection ball positioning mark instrument.

According to the present invention, safety of a spine surgery performed by a surgeon and a surgery quality can be effectively improved, usage of a radioactive medical image in a surgery can be reduced, and free radiation absorption of health care providers can be reduced. In addition, a medical image navigation technology is a core basis of the future intelligent surgery assistant system. A combination of a surgery mechanical arm and a high-focusing penetrative treatment device (an HIFU, a Gamma Knife, or proton therapy) in the future can further implement high precision treatment and reduce postoperative complications and affects.

In conclusion, this application is merely an implementation manner or an embodiment of a technical solution for resolving a problem and is not intended to limit the patent implementation scope of this application. Any equivalent modification and decoration made in accordance with the patent application scope of this application or within the patent scope of this application shall fall within the patent scope of this application.

What is claimed is:

1. A surgery navigation system for a spine surgery, comprising:
a positioning module, comprising:
a transceiver unit, configured to transmit a frequency modulated signal and receive a positioning mark frequency signal and an instrument frequency signal;
multiple positioning marks, which are separately disposed on each vertebra of a spine, wherein each positioning mark is configured to transmit the positioning mark frequency signal to the transceiver unit after receiving the frequency modulated signal; and
a surgical instrument, configured to transmit the instrument frequency signal to the transceiver unit after receiving the frequency modulated signal; and
a processing unit, configured to obtain a positioning mark distance between the positioning marks and the transceiver unit by using an algorithm through calculation according to a difference between the positioning mark frequency signal and the frequency modulated signal, obtain space coordinates of the spine through calculation according to the positioning mark distance, obtain an instrument distance between the surgical instrument and the transceiver unit by using the algorithm through calculation according to a difference between the instrument frequency signal and the frequency modulated signal, obtain space coordinates of the instrument through calculation according to the instrument distance, and perform a surgery navigation operation according to the space coordinates of the spine and the space coordinates of the instrument.

2. The surgery navigation system according to claim 1, wherein the algorithm is a frequency modulated continuous wave (FMCW) positioning algorithm, the processing unit obtains the positioning mark distance through calculation according to the difference between the positioning mark frequency signal and the frequency modulated signal that are received at the same time, and the processing unit obtains the instrument distance through calculation at the same time according to the difference between the instrument frequency signal and the frequency modulated signal.

3. The surgery navigation system according to claim 1, wherein the positioning marks further comprise an identifier, the surgical instrument further comprises an instrument identifier, the transceiver unit is configured to receive the identifier and the instrument identifier, the processing unit establishes a correspondence between the identifier and the space coordinates of the spine, and the processing unit establishes a correspondence between the instrument identifier and the space coordinates of the instrument.

4. The surgery navigation system according to claim 1, wherein the processing unit obtains the space coordinates of the spine through calculation according to the positioning mark distance by using a triangulation method, and the processing unit obtains the space coordinates of the instrument through calculation according to the instrument distance by using the triangulation method.

5. The surgery navigation system according to claim 1, wherein each positioning mark further comprises at least two antennas, after receiving the frequency modulated signal, the at least two antennas transmit two positioning mark frequency signals to the transceiver unit, and the processing unit obtains distances between the two antennas and the transceiver unit through calculation by using the two positioning mark frequency signals, so as to obtain an angle of the positioning mark through calculation.

6. The surgery navigation system according to claim 1, wherein the surgical instrument further comprises at least two instrument antennas, after receiving the frequency modulated signal, the at least two instrument antennas transmit two instrument frequency signals to the transceiver unit, and the processing unit obtains distances between the two instrument antennas and the transceiver unit through calculation by using the two instrument frequency signals, so as to obtain an angle of the surgical instrument through calculation.

7. The surgery navigation system according to claim 4, comprising at least two transceiver units, which are separately disposed at a periphery of the spine, and the processing unit obtains two corresponding positioning mark distances through calculation according to two positioning mark frequency signals received by the two transceiver units and then obtains the space coordinates of the spine through calculation by using the triangulation method.

8. The surgery navigation system according to claim 4, wherein there are at least two transceiver units, which are separately disposed at a periphery of the surgical instrument, and the processing unit obtains two corresponding instrument distances through calculation according to two instrument frequency signals received by the two transceiver units and then obtains the space coordinates of the instrument through calculation by using the triangulation method.
